(19) United States
(12) Patent Application Publication
(10) Pub. No.: US 2010/0095965 A1
(43) Pub. Date: Apr. 22, 2010

(54) Enhanced means for regulating intrathoracic pressures

(76) Inventor: S. David Piper, Carmichael, CA (US)

Correspondence Address: David Piper, P.O. Box 993, Carmichael, CA, 95609, US

(21) Appl. No.: 12/589,015

(22) Filed: Oct. 16, 2009

Related U.S. Application Data
(60) Provisional application No. 61/196,430, filed on Oct. 16, 2008.

(51) Int. Cl. A61M 16/20

(52) U.S. Cl. 128/205.24

(57) ABSTRACT

The present invention relates generally to devices and methods for finite control and regulation of patient intrathoracic pressures, and more specifically, to devices and methods that are finitely adjustable within a range set by an operator for regulating a patient intrathoracic pressures during repeated cycling events (i.e. respiration). The enhanced means includes a dual area valve on an exhalation and/or inhalation port of a device such that the valve is biased against the pressure necessary to evacuate and/or inflate the lungs of that patient by at least a partial volume thereof. The enhanced means for regulating intrathoracic pressure are applicable in a number of medically important therapies, including but not limited to, conditioning of pulmonary systems for acclimation to altered environmental conditions, reconditioning of pulmonary system after operating in a diminished state, and application in cardiopulmonary resuscitation procedures.

### CROSS-REFERENCE TO RELATED APPLICATIONS

This application claims the benefit under 35 U.S.C. 119(e) of U.S. provisional application Ser. No. 61/196,430 filed Oct. 16, 2008, which is incorporated by reference herein in its entirety.

### STATEMENT REGARDING FEDERALLY SPONSORED RESEARCH

Not Applicable

### BACKGROUND OF THE INVENTION

The present invention relates generally to devices and methods for finite control and regulation of patient intrathoracic pressures and patient respiration, and more specifically, to devices and methods that are finitely adjustable within a range set by an operator for regulating a patient intrathoracic pressure during repeated cycling events (i.e. natural or artificial respiration). The enhanced means for regulating intrathoracic pressure during patient respiration are applicable in a number of medically important therapies, including but not limited to, conditioning of pulmonary systems for acclimation to altered environmental conditions, reconditioning of pulmonary system after operating in a diminished state, and application in cardiopulmonary resuscitation procedures.

Intrathoracic pressure as related to a patient is a measure of performance generated in the thoracic region defined in the upper chest as the volume between the posterior T10 spinous process, the anterior xiphoid process of the ribcage and bounded distally by the diaphragm (Clemente, C. D. (1981). Anatomy). The lungs of the patient are encircled by the thoracic region. As autonomous or artificial breathing occurs, during inspiration the diaphragm descends, the ribcage elevates, and the intrathoracic region volumes increases thereby creating a decreased intrathoracic pressure. Gas exterior to the patient (either environmental or artificially introduced) is drawn into the lungs as a result of this decreased or negative pressure. Upon expiration, the diaphragm ascends; the ribcage descends causing a decrease in intrathoracic volume and an increase in intrathoracic pressure. Gas within the patient's lung is then at a higher pressure than the environment, and thus the gas is expelled from the lung.

Under normal circumstances (i.e. a patient with a healthy respiratory system) the cycling of positive and negative pressures within the intrathoracic region occurs in a continuous, regular pattern as a result of normal respiration. However, this pattern can be interrupted in terms of frequency and/or amplitude due to such effectors as induced respiratory stress (e.g. exercise), diminished respiratory capacity (e.g. disease or injury) and compromised respiratory performance (e.g. cardiopulmonary collapse). While the first effecter, induced respiratory stress, is focused on an incremental improvement in respiratory performance, the latter two effectors are emergent in nature and require medical attention in order to sustain sufficient respiration and maintain patient viability. To counter each of these effectors, it is desirable to introduce a means into the patient respiratory tract such that the degree and duration of positive and/or negative pressures attained during respiration cycling is finitely regulated.

An incremental improvement in respiratory performance is desirable by individuals who require an enhanced ability to cycle oxygen into their systems. Athletes who subject their systems to sudden or prolonged stress can benefit from artificially restricted or altered respiratory environments. Use of an artificially restricted respiratory environment causes the individual's intrathoracic region to develop with higher capacities, greater musculature, and quicker recovery times. Therefore, when an athlete trains in an artificially restricted respiratory environment, and that environment is then removed, the individual then enjoys a higher respiratory capacity (“Update in the understanding of respiratory limitations to exercise performance in fit, active adults”, Dempsey, et al., Chest 2008, September; 134(3), incorporated by reference herein its entirety). Such incremental improvements can also be desirable to individuals who must operate in environments where atmospheric pressure is at extremes, such as deep sea diving and high altitude climbing.

Diminished respiratory capacity is evident in patients, which have either existing damage or disease in the lung or other elements of the respiratory, pulmonary, and circulatory tracts or injury caused to an element of the system by accident or surgical intervention. In the situation wherein the respiratory system is damaged, it can be desirable to integrate into a gas supply a finite ability to control the positive and negative pressures created in the intrathoracic region so as to minimize further damage, improve performance, and assist in reconditioning of the system. Numerous modes of injury and damage to the respiratory system exists, as generally taught in the following published citations, incorporated by reference herein in their respective entireties: “Clinical review: Positive end-expiratory pressure and cardiac output”, Luecke, et al., Critical Care. 2005; 9(6); “Physiological changes occurring with positive pressure ventilation”, Robb, Intensive Critical Care Nurse 1997 October; 13(5); and, “Is there a best way to set positive expiratory-end pressure for mechanical ventilatory support in acute lung injury?”, Maclntyre, Clinical Chest Med. 2008 June; 29(2).

Compromised respiratory performance is defined as failure of the respiratory system to cycle, often as an element of complete cardiopulmonary collapse. Cardiopulmonary collapse, or sudden cardiac arrest, is a major cause of death worldwide and is the result of a variety of circumstances, including heart disease and significant trauma. In the event of a cardiac arrest, a rapid and appropriate response is essential in order to improve a patient's chance of survival by at least partially restoring the patient's respiration and blood circulation. External chest compression technique generally referred to as cardiopulmonary resuscitation (CPR) is the most common means of attaining partial respiration and circulation in a patient.

Intrathoracic pressure is momentarily increased through application of external force as part of the CPR procedure. An increase in intrathoracic pressure induces blood movement from the region of the heart and lungs towards the peripheral arteries. Such pressure increase partially restores the patient's circulation. Traditional CPR is performed by actively compressing the chest by direct application of an external pressure to the chest. After active compression, the chest is allowed to expand by its natural elasticity which causes expansion of the patient's chest wall. This expansion allows some blood to reenter the cardiac chambers of the heart. The procedure as described, however, is insufficient to induce sufficient respiration in the patient. To attain respiration, conventional CPR also requires periodic ventilation of the patient. This is commonly accomplished by mouth-to-mouth technique or by using positive-pressure devices, such as a self-inflating bag, which relies on squeezing an elastic bag to deliver gas into the patient's respiratory system.

With CPR, and other similar techniques, an increase in the amount of venous blood flowing into the heart and lungs from the peripheral venous vasculature is desirable to increase the volume of oxygenated blood leaving the thorax during the subsequent compression phase. It would therefore be desirable to provide improved methods and apparatus for enhancing venous blood flow into the heart and lungs of a patient from the peripheral venous vasculature as well as enhancing blood leaving the thorax during CPR. Further, it would be particularly desirable to provide techniques which would enhance oxygenation and increase the total blood return to the chest during the decompression step of CPR and increase the total blood flow leaving the thorax during the compression set of CPR.

Improvement in oxygenation and blood flow can be accomplished by regulating intrathoracic pressure, thereby amplifying the total intrathoracic pressure swing. U.S. Pat. Nos. 6,986,349; 6,604,523; 6,526,973; and 6,425,393 to Lurie et al., each incorporated by reference it their respective entireties, teach to use of a valve type impingement in the respiratory tract of a patient utilizing continuous vacuum application. Upon analysis of such a device as described by Lurie, et al., it is found that by continuous application of a set vacuum, an initial enhancement is obtained in a first CPR compression cycle, but the benefit is diminished over subsequent cycles as the set vacuum effectively establishes and maintains the intrathoracic cavity to a finite lower pressure point.

There exists a need for a means to finitely regulate the intrathoracic pressure of a patient, which is readily applied to a patient and offers improved means for controlling the positive and/or negative pressures achieved in the intrathoracic region and the rates by which those pressures are developed.

### SUMMARY OF THE INVENTION

The present invention relates generally to devices and methods for finite control and regulation of patient intrathoracic pressures, and more specifically, to devices and methods that are adjustable within a range set by an operator for regulating patient intrathoracic pressures during repeated cycling events. The enhanced means includes a dual area valve on a port of an cardiac assist assembly such that the valve is biased against the pressure necessary to evacuate the lungs of that patient by at least a partial volume thereof. The dual area valve requires an initial threshold pressure be exceeded, which in turn opens the valve, and the valve remains in an open position until a lower reset pressure is achieved. The enhanced means further includes various valve types and mechanisms on an inhalation or exhalation port of a separate or same device assembly such that the valve is biased against the pressure necessary to inflate or exhaust the lungs of that patient by at least a partial volume thereof. The enhanced means for regulating intrathoracic pressure are applicable in a number of medically important therapies, including but not limited to, conditioning of pulmonary systems for acclimation to altered environmental conditions, reconditioning of pulmonary system after operating in a diminished state, and application in cardiopulmonary resuscitation procedures.

In a first embodiment, a patient connector is in fluid communication with a valve assembly, wherein the valve assembly comprises a dual area valve piston associated with and biased against an exhalation port of the assembly and a conventional check valve is associated and biased against an inhalation port of the assembly which allows for atmospheric or controlled gas introduction. The dual area valve piston base engages upon an extended shoulder of a dual area valve base such that a seal is created between a face of the dual area valve piston base and the extended shoulder. The surface area described by the internal diameter of the extend shoulder defines a closed position area. The dual area valve piston base has a separate area defined by the total surface area of the piston face, which is termed the open position area. The dual area valve piston base is maintained against the extend shoulder of the dual area valve base by the force exerted by a biasing means (e.g. helically wound spring), though it is within the purview of the present invention that the biasing means can be mechanically or electronically adjusted through manual, semi-automated and fully automated processes. The biasing means is retained in the assembly by a dual area valve top, which itself includes a plurality of atmospheric vents. As pressure from a chest compression (or induced tidal volume) through a patient connector through the dual area valve base and against dual area valve piston base, the biasing means maintains the dual area valve piston base closed until an initial threshold pressure is exceeded. Once the pressure exceeds the force exerted by biasing means, the dual area valve piston base will translate from a closed to an open condition, and will remain open until sufficient pressure is dissipated as to allow biasing means to return the dual area valve piston base to a closed position against extended shoulder of dual area valve base. The rate at which the pressure drops from the high opening pressure to the lower closing pressure is regulated in part by the restrictive values of cross-seal vents in the dual area valve base.

It is critical to note that the operation of the aforementioned valve assembly allows for an initial higher pressure to cause the piston to open and to then subsequently close based on a second lower pressure being achieved. By setting the trigger pressure and the reset pressure based on patient parametrics, including lung capacity, the pressure within the intrathoracic region can be specifically regulated. Those skilled in the art can appreciate that a number of alternate piston and biasing schemes could be employed without departing from the dual area nature of the invention. By impeding exhalation gas flow, the present invention specifically regulates the application and retention of pressure within the thorax. This is superior to normal CPR techniques without the invention as in such a case the CPR compression would primarily be functioning to simply push out gas in the patient's lungs and thus would result in less force being applied to induce blood flow leaving the thorax. Furthermore, the invention has the further advantage of providing feedback to the clinician or operator on whether sufficient chest compression has been supplied (as indicated by the piston moving from the closed to the open position) and providing a degree of assurance that excessive force has not been applied as the device can be set specifically for elderly and pediatric patients.

In a second embodiment, a patient connector is in fluid communication with a valve assembly, wherein the valve assembly comprises a dual area valve piston associated with and biased against an exhalation port of the assembly (as in the first embodiment) and a constant vacuum valve associated with and biased against an inhalation port of the assembly which creates a controlled negative pressure during gas inhalation. The constant vacuum valve comprises a simple biased valve wherein the biasing means may include a valve spring, which when associated with a vacuum source, will allow for pressure reduction when activated. This alternate assembly includes a dual area valve biased against exhalation as described in the first embodiment, wherein the second embodiment will function similarly during chest compression. During release of the chest compression, the constant vacuum valve biased in the inhalation port of the this embodiment will not open until a sufficient vacuum has been produced in the intrathoracic region to overcome the biasing force of the vacuum valve spring, at which point flow will occur at the set vacuum pressure. Providing vacuum in this manner serves to provide greater pressure on intrathoracic region as a result of the ambient pressure of the surrounding environment and thus increases blood flow to the heart and lungs from the peripheral venous vasculature. By setting the constant vacuum level, the trigger pressure, the reset pressure, and the decline rate from the trigger pressure to the reset pressure based on patient parametrics, including lung capacity, the pressure within the intrathoracic region can be specifically regulated and greater flow into and out of the thoracic region can be realized.

In a third embodiment, a patient connector is in fluid communication with a compound valve assembly, wherein the compound valve assembly comprises a first dual area valve piston associated with and biased against an exhalation port of the assembly (as described in the first embodiment) and a second dual area vacuum valve associated with and biased against an inhalation port of the assembly. This third embodiment includes the same dual area valve piston that is biased against an exhalation port as described in the first embodiment, and as such the third embodiment will function the same as the first embodiment during chest compression. As the chest compression is released, the dual area vacuum valve remains closed until sufficient vacuum pressure is generated to overcome a biasing force provided by a dual area valve vacuum spring, at which time the dual vacuum valve opens and remains open until a lower closing vacuum pressure is reached. Providing vacuum in this manner serves to provide greater pressure on intrathoracic region as a result of the ambient pressure of the surrounding environment and thus increases blood flow to the heart and lungs from the peripheral venous vasculature. The use of a dual area vacuum valve allows for a decrease in vacuum pressure prior to the next chest compression, thus allowing gas to re-enter the thoracic cavity to return to a more normal physiological condition and re-setting the stage for a subsequent and more successful chest compression (i.e. more blood flow) than may be realized by a constant vacuum only methodology.

In a fourth embodiment, a patient connector is in fluid communication with a flapper valve assembly, wherein the flapper valve assembly comprises a simple restriction biased against an exhalation port of the assembly and a dual area valve vacuum piston associated with and biased against an inhalation port of the assembly. The simple restriction associated with the exhalation port of the assembly allows for a pressure release from the intrathoracic region at a constant rate during chest compression. As the chest compression is released, the dual area vacuum valve remains closed until sufficient vacuum pressure is generated to overcome a biasing force provided by a dual area valve vacuum spring, at which time the dual vacuum valve opens and remains open until a lower closing vacuum pressure is reached. Providing vacuum in this manner serves to provide greater pressure on intrathoracic region as a result of the ambient pressure of the surrounding environment and also thus increases blood flow to the heart and lungs from the peripheral venous vasculature. This fourth embodiment includes additional vacuum control over the method described in the second embodiment as it provides means for a higher peak vacuum pressure, resulting in more cardiac blood flow than a system without such control means. The use of a dual area vacuum valve allows for a decrease in vacuum pressure prior to the next chest compression, thus allowing gas to re-enter the thoracic cavity to return to a more normal physiological condition and re-setting the stage for a subsequent and more successful chest compression cycle (i.e. more blood flow) than may be realized by a constant vacuum only methodology.

Although a constant vacuum only methodology has been shown to be useful, the current invention is more effective based on a theory that additional blood flow into the heart during a finite vacuum phase occurs prior to a subsequent chest compression. In contrast to a constant vacuum methodology, there is no added benefit of holding vacuum throughout the entire decompression phase of CPR, and doing so impedes the success of subsequent chest compressions. The current invention provides suitable magnitude and duration of vacuum necessary to realize the added blood flow of the initial chest compression of a constant vacuum methodology, and then beneficially allows the thoracic cavity to return to ambient pressure (prior to the subsequent chest compression), setting the stage for equally effective subsequent chest compressions. To present this point in alternate wording: a constant vacuum methodology is primarily effective on the first chest compression and not on subsequent chest compressions, until such time that a breath is delivered to the patient and at which point the thoracic cavity is effectively reset to ambient pressure by this manual and deliberate action. The current invention is as effective on the first chest compression, and equally effective on every subsequent chest compression thereafter. The current invention requires no manual resetting to ambient pressure, and is therefore more suited to CPR situations in which there is only one rescuer, when the CPR standards direct focus onto chest compressions and not breaths.

Each of the above embodiments can be used with a sealing mask, endotracheal tube, or any other equivalent respiratory patient engagement or sealing means.

A further embodiment includes incorporating an embodiment described above with a self inflating bag, commonly referred to as a manual resuscitator or an “ambu bag.” As described in the prior art, the typical arrangement is for an “ambu bag” is to have a self inflating bag connected to a valved assembly which includes 3 ports: 1. for connection to the bag; 2. for connection to the patient; and 3. an outlet to the ambient environment. Within the valved assembly is a “duck bill” valve or equivalent arrangement such that when pressure is applied to the bag, the ambient outlet port is closed to the patient port and gas is caused to pass from the bag, through the valve and to the patient. When the self-inflating bag is released, the valve prevents the flow of gas from the patient to the bag and directs it to the ambient outlet port.

A further embodiment includes incorporating an embodiment described above with a self inflating bag such that a constant or dual vacuum valve is placed between the bag and the valve assembly and a dual area piston is placed onto the ambient port. In this manner a combination CPR breathing rescue device is able to provide all the advantages and functions as previously described in embodiment three. Upon the squeezing of the bag for providing inhalation to the patient, gas would pass through the dual vacuum piston valve and onto the patient. Because the bag valve closes the outlet ambient port when the bag is squeezed, the rescuer would be able to provide whatever gas was necessary to the patient without triggering the dual area piston. Upon release of the bag the patient pressure would trigger the dual area piston and the normal advantages of CPR would still be available.

According to the present invention, use of a finitely regulated dual area valve assembly can be readily employed for increasing cardiopulmonary circulation induced by chest compression and decompression when performing cardiopulmonary resuscitation is provided. Particularly advantageous is in the use of a dual area valve assembly biased against patient exhalation and/or inhalation and any one of the described (equivalent/alternate) control means biased against patient exhalation and/or inhalation is that “locked pressure windows” can be created transiently in the cycling of the positive and negative pressures formed in the intrathoracic region of a patient. These “locked pressure windows” are points where due to biasing of the respective control means on the exhalation and inhalation ports of a valve assembly attached to the respiratory system of a patient, either inhalation or exhalation can occur within a finite set of conditions and therefore a set pressure, either positive or negative, is retained with the intrathoracic region. The methods and devices may be used in connection with any generally accepted CPR methods or with active compression-decompression (ACD) CPR techniques. When a valve assembly in accordance with the present invention is used with CPR methods, the transient “locked pressure windows” automatically coincide with steps in the CPR method such that less force is lost in movement of air volumes from the patient's thoracic region and incremental pressure gains are achieved in inducing circulation of oxygenated blood in the patient (measured as flow).

Cardiopulmonary circulation is increased according to the invention by impeding air flow into and/or out of a patient's lungs during the compression and/or decompression phase. This increases the magnitude and prolongs the duration of positive and/or negative intrathoracic pressure during compression and the subsequent decompression of the patient's chest and result in increases of venous blood flow into the heart and lungs from the peripheral venous vasculature during decompression and also results in increases in oxygenated blood leaving the thorax during compression. Thus the present invention results in the greater inflow and outflow of blood through the heart and lung corresponding with the initiation of compression and decompression accompanying CPR rather than the diminished blood flow and the increased flow of gases coming in and out of the lung that would result without the invention. As the inventive concept provides for a return to a baseline lung pressure, the invention has the further advantage over other technologies of still allowing gas exchange as a result of CPR and works harmoniously with various ventilation technologies and artificial breathing techniques.

In a specific embodiment, impeding the air flow into the patient's lungs is accomplished by altering ventilation during the decompression phase of CPR through use of a valve assembly having the ability to finitely regulate the intrathoracic pressure of the patient. The dual area vacuum valve is biased to open and permit the inflow of air when the intrathoracic pressure falls below a threshold level. In order to properly ventilate the patient, as opposed to simple CPR decompression, the invention allows for periodically ventilating the patient by providing a positive pressure of gas into the gas inlet of the dual area vacuum valve whether by use of a ventilator technology, manual resuscitator, or other artificial breathing techniques.

When performing cardiopulmonary resuscitation to enhance circulation according to the invention, an operator compresses a patient's chest to force blood out of the patient's thorax. The patient's chest is then decompressed to induce venous blood to flow into the heart and lungs from the peripheral venous vasculature either by actively lifting the chest (via ACD-CPR) or by permitting the chest to expand due to its own elasticity (via conventional CPR). During the decompression step, air flow is impeded from entering into the patient's lungs which enhances negative intrathoracic pressure and increases the time during which the thorax is at a lower pressure than the peripheral venous vasculature. Thus, venous blood flow into the heart and lungs from the peripheral venous vasculature is enhanced during decompression as a result of enhanced venous return rather than from inflow of air via the trachea. In a particular embodiment, compression and decompression of the patient's chest may be accomplished by pressing an applicator body against the patient's chest to compress the chest, and lifting the applicator to actively expand the patient's chest.

Any of the above embodiments may further include one or more CPR assistant devices into the dual area valve piston assembly, wherein one or more visual and/or aural signals are provided to the operator of the device for effectively conducting CPR (i.e. pace or rate, measure of applied force) and/or patient condition (i.e. pulse, return of autonomic function/respiratory response).

Other features and advantages of the present invention will become readily apparent from the following detailed description, the accompanying drawings, and the appended claims.

### BRIEF DESCRIPTION OF THE DRAWINGS

The invention will be more easily understood by a detailed explanation of the invention including drawings. Accordingly, drawings which are particularly suited for explaining the inventions are attached herewith; however, it should be understood that such drawings are for descriptive purposes only and as thus are not necessarily to scale beyond the measurements provided. The drawings are briefly described as follows:

FIG. 1 is an upper exploded perspective view of a valve assembly in accordance with a first embodiment of the present invention;

FIG. 2 is a lower exploded perspective view of a valve assembly in accordance with a first embodiment;

FIG. 3 is an end view of a valve assembly in accordance with a first embodiment;

FIG. 4 is a cross-sectional view taken along line B-B in FIG. 3;

FIG. 5 is a front side view of a valve assembly in accordance with a first embodiment;

FIG. 6 is a back side view of a valve assembly in accordance with a first embodiment;

FIG. 7 is a top side view of a valve assembly in accordance with a first embodiment;

FIG. 8 is a bottom side view of a valve assembly in accordance with a first embodiment;

FIG. 9 is a right end view of a valve assembly in accordance with a first embodiment;

FIG. 10 is an upper exploded perspective view of a constant vacuum valve in accordance with a second embodiment of the present invention;

FIG. 11 is a left end view of a constant vacuum valve in accordance with a second embodiment;

FIG. 12 is a cross-sectional view of a constant vacuum valve taken along line A-A in FIG. 12;

FIG. 13 is a front side view of a valve assembly in accordance with a second embodiment;

FIG. 14 is a back side view of a valve assembly in accordance with a second embodiment;

FIG. 15 is a top side view of a valve assembly in accordance with a second embodiment;

FIG. 16 is a bottom side view of a valve assembly in accordance with a second embodiment;

FIG. 17 is an upper exploded perspective view of a valve assembly in accordance with a third embodiment of the present invention;

FIG. 18 is side view of a dual vacuum piston assembly in accordance with a third embodiment of the present invention;

FIG. 19 is cross-sectional view of a dual vacuum piston assembly along line C-C in FIG. 19;

FIG. 20 is a lower perspective view of a secondary dual vacuum piston assembly in accordance with a third embodiment;

FIG. 21 is an upper perspective view of a valve assembly in accordance with a third embodiment;

FIG. 22 is an front side view of a valve assembly in accordance with a third embodiment;

FIG. 23 is a left end view of a valve assembly in accordance with a third embodiment;

FIG. 24 is an upper exploded perspective view of a valve assembly in accordance with a third embodiment;

FIG. 25 is upper perspective view of a valve assembly in accordance with a fourth embodiment of the present invention;

FIG. 26 is top view of a valve assembly in accordance with a fourth embodiment;

FIG. 27 is a bottom view of a valve assembly in accordance with a fourth embodiment;

FIG. 28 is an front side view of a valve assembly in accordance with a fourth embodiment;

FIG. 29 is an back side view of a valve assembly in accordance with a fourth embodiment; and

FIG. 30 is a left end view of a valve assembly in accordance with a fourth embodiment of the present invention.

### LIST OF REFERENCE NUMERALS

With regard to reference numerals used, the following numbering are applied throughout the drawings: patient connector 1, check valve body 2, check valve flapper 3, dual area valve base 4, dual area valve piston base 5, dual area valve piston seal 6, dual area valve piston top 7, dual area valve top 8, biasing means 9, constant vacuum valve base 10, constant vacuum valve top 11, constant vacuum valve spring 12, constant vacuum valve piston 13, dual vacuum valve base 14, dual vacuum valve top 15, dual vacuum piston base 16, dual vacuum valve piston seal 17, dual vacuum valve piston top 18 and dual vacuum valve spring 19, exhalation flapper valve 21, cross seal vent 22, patient port 23, inhalation port 24, atmospheric vent 25, dual area exhalation valve assembly 30, constant vacuum valve assembly 40, dual area vacuum valve assembly 50.

### DETAILED DESCRIPTION OF SPECIFIC EMBODIMENTS

While the present invention is susceptible of embodiment in various forms, there is shown in the drawings and will hereinafter be described a presently preferred embodiment of the invention, with the understanding that the present disclosure is to be considered as an exemplification of the invention, and is not intended to limit the invention to the specific embodiment illustrated. Herein, the term “exhalation” is used as to describe any event, whether voluntary on the part of the patient or not, which results in any amount of expelled gas or pressure from the patient. Similarly, the term “inhalation” is used to describe any event that results in any receipt of gas by the patient, or a vacuum pressure relative to ambient air.

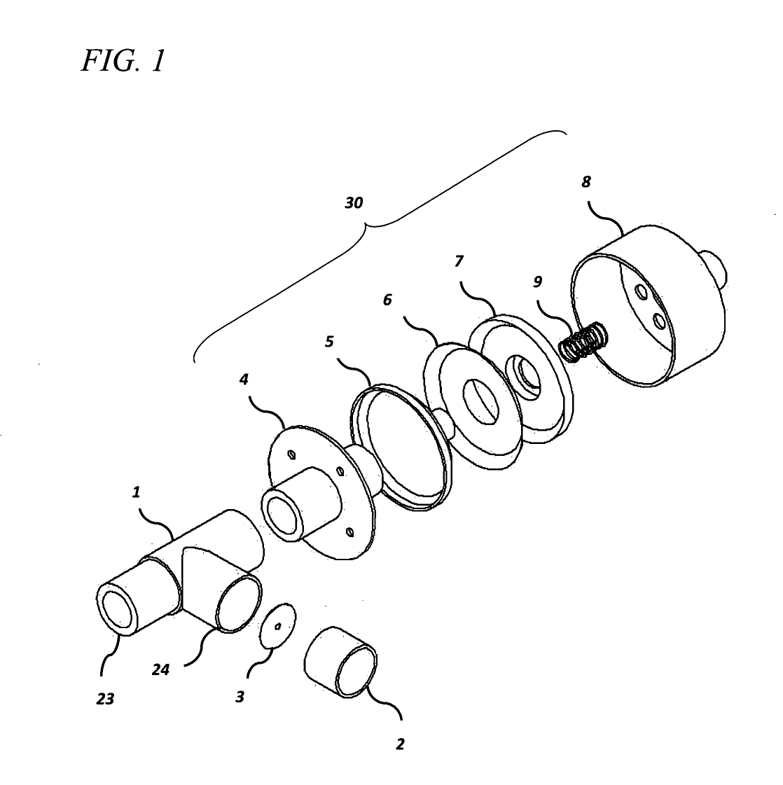

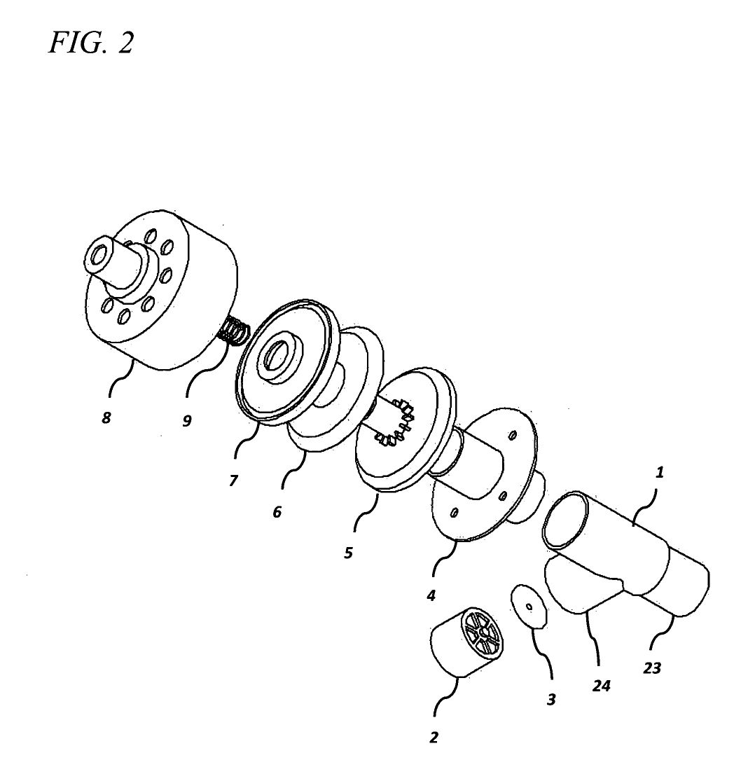

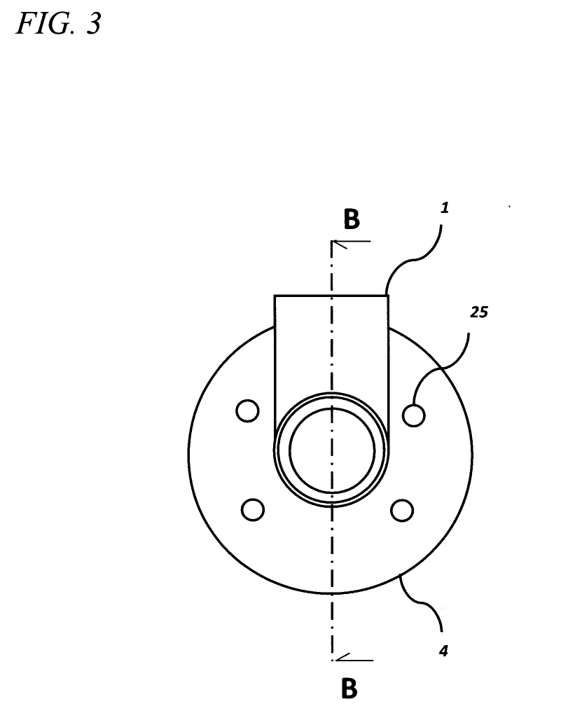

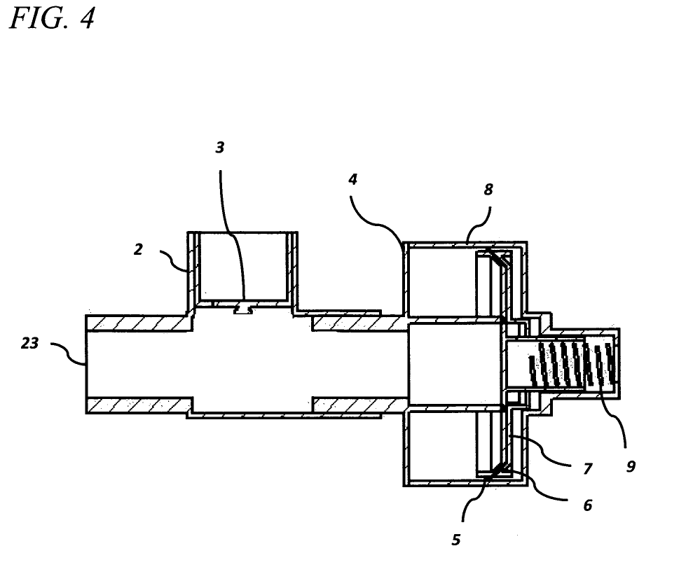

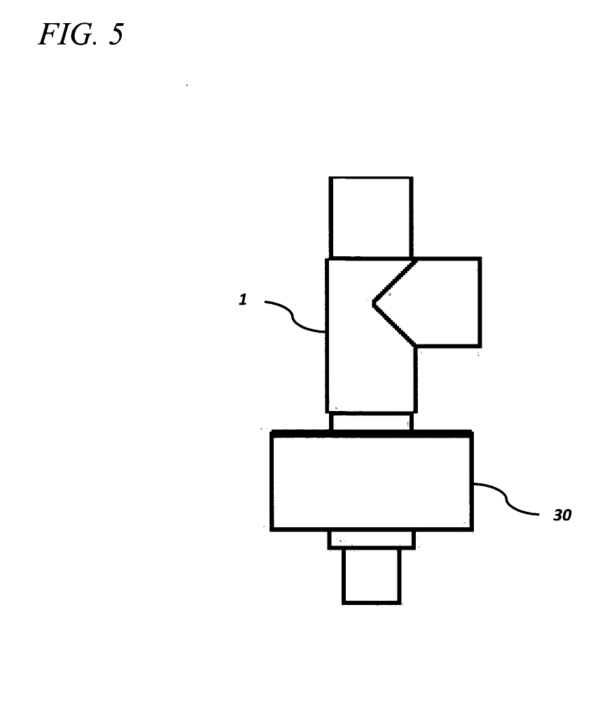

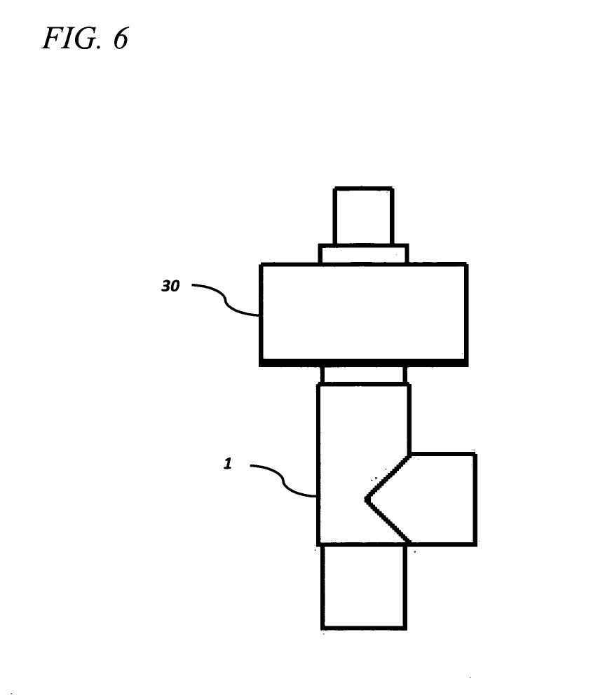

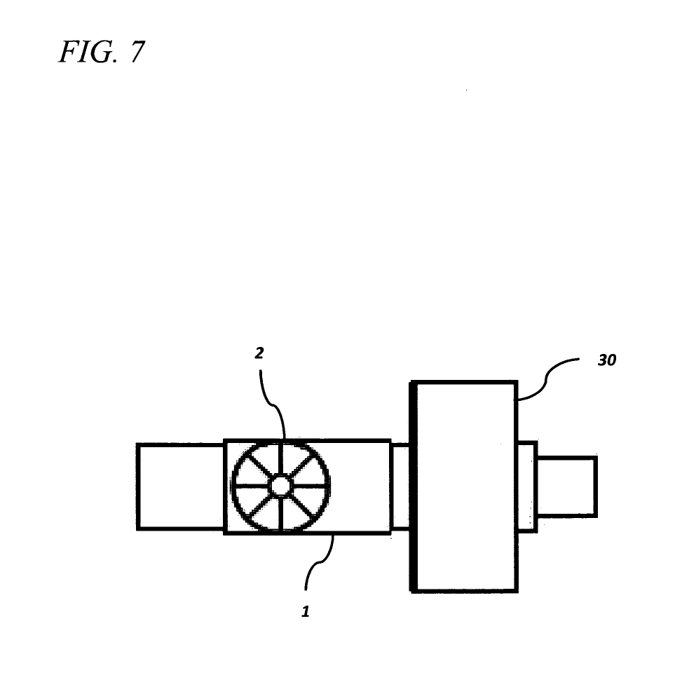

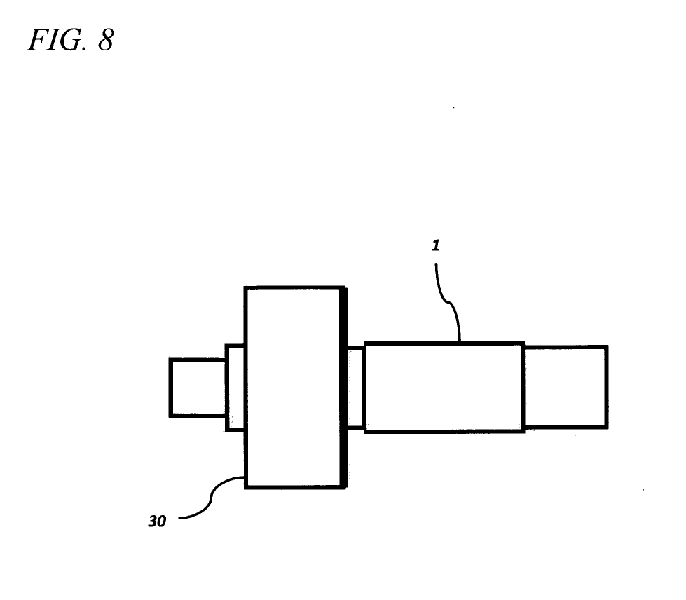

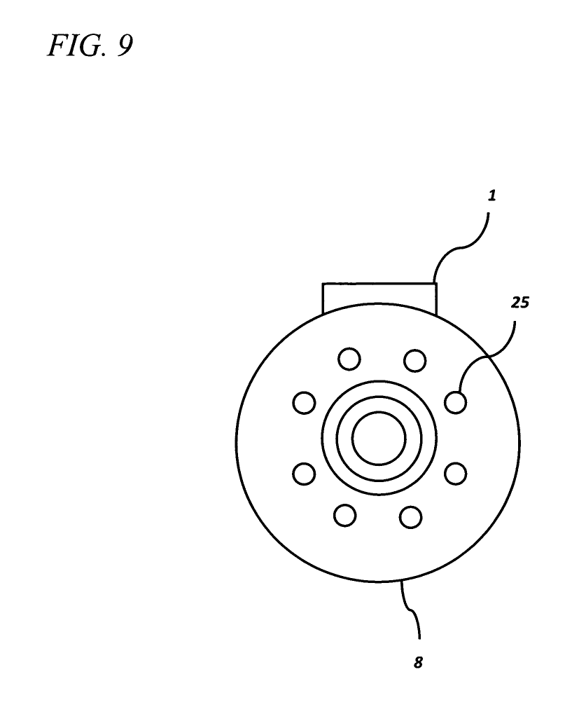

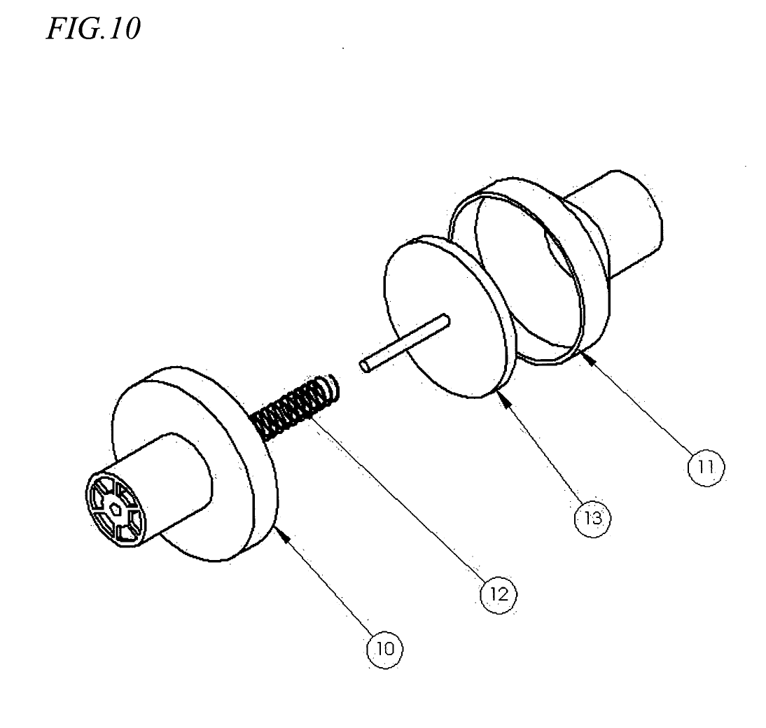

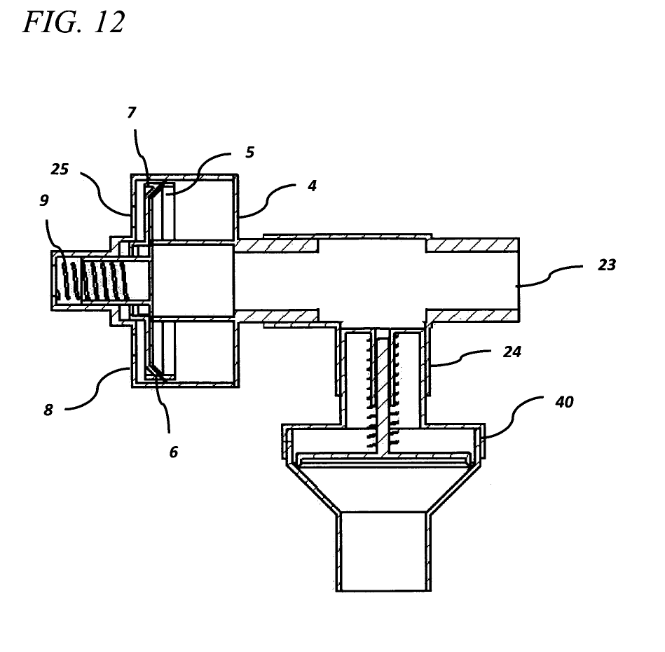

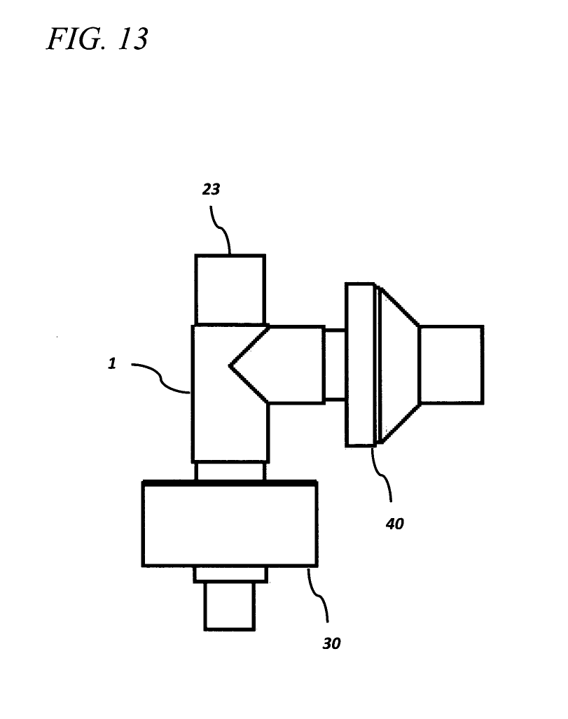

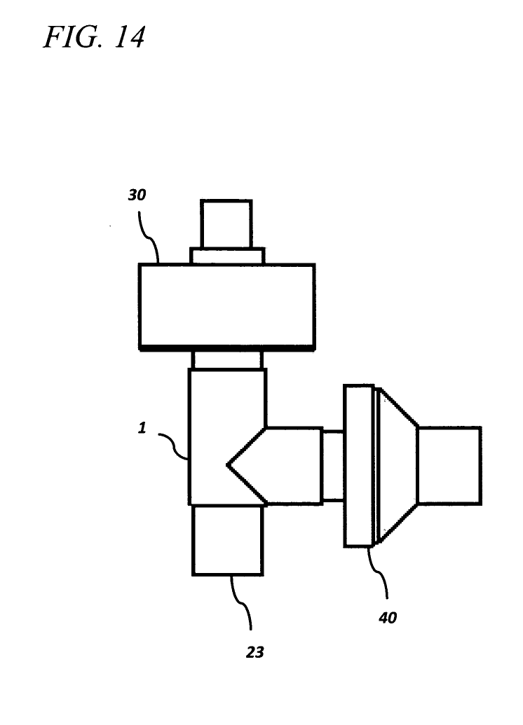

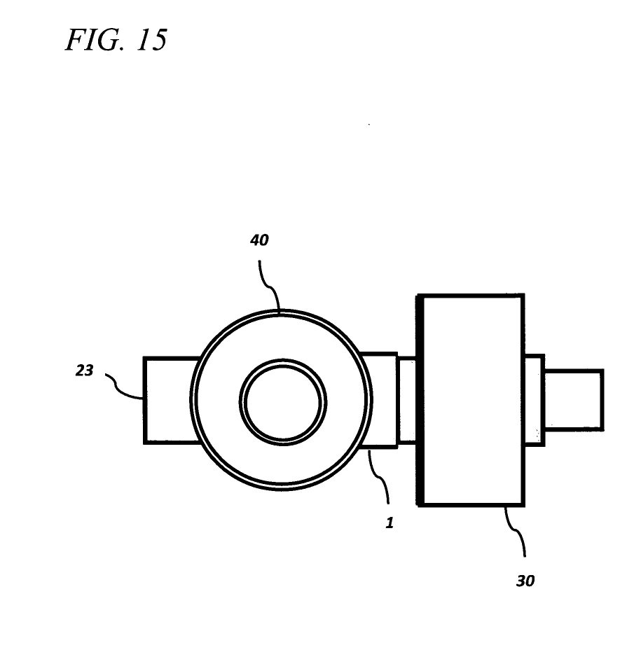

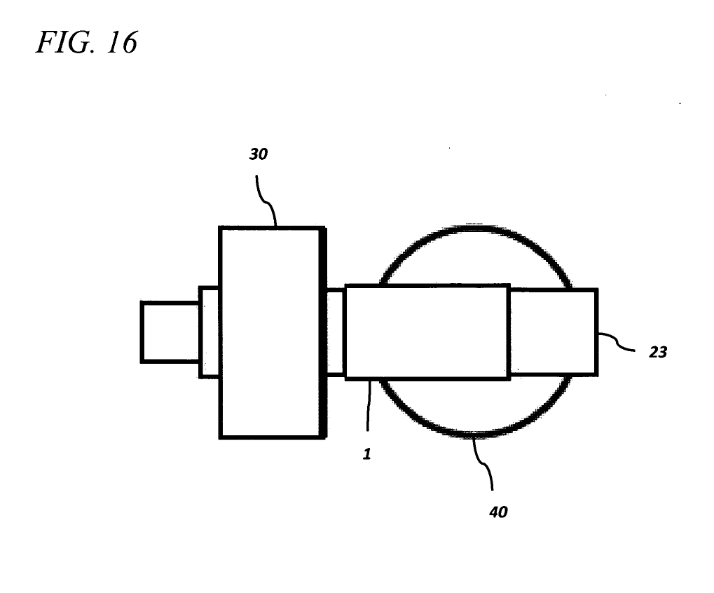

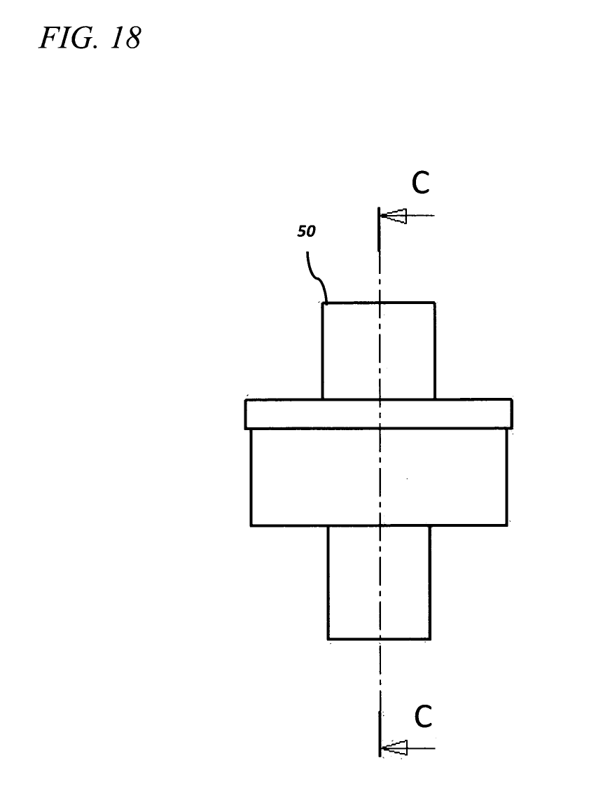

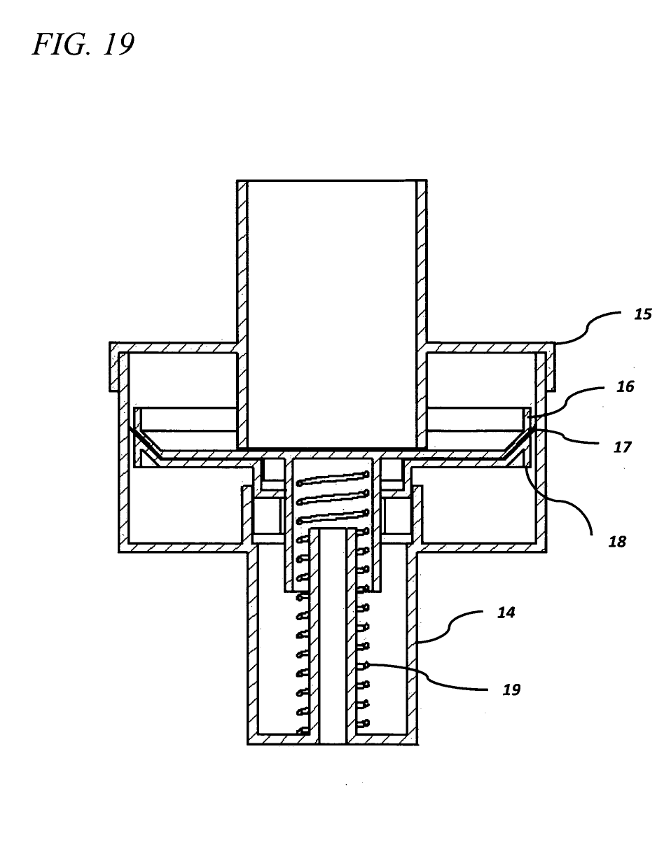

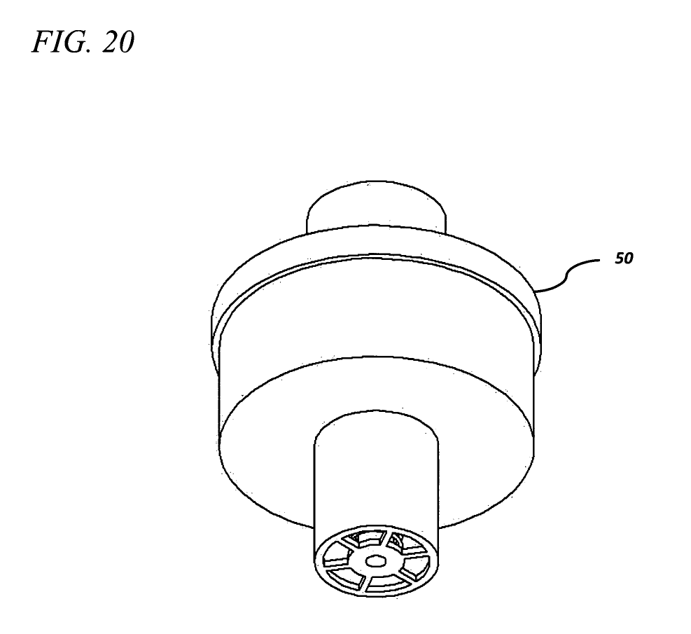

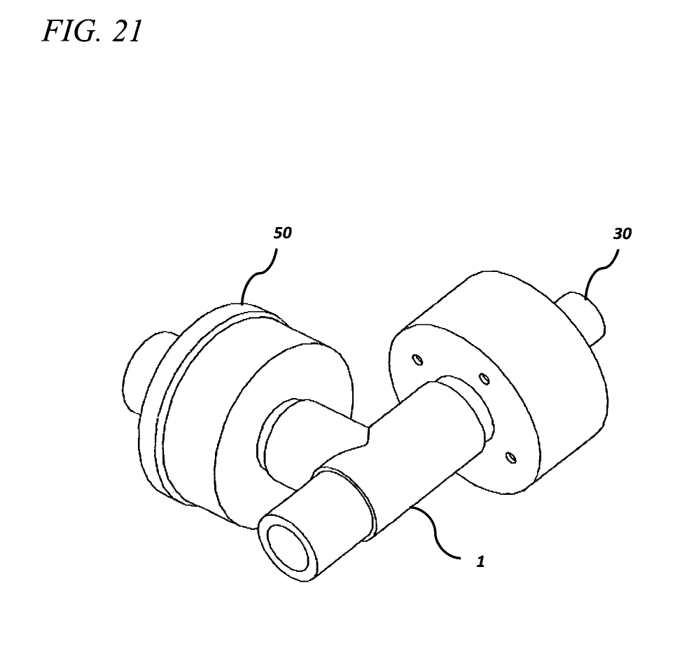

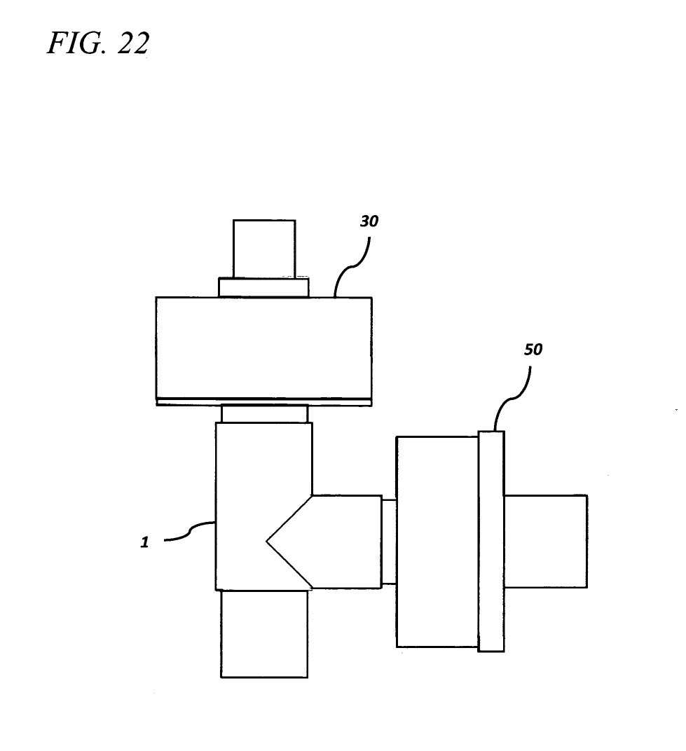

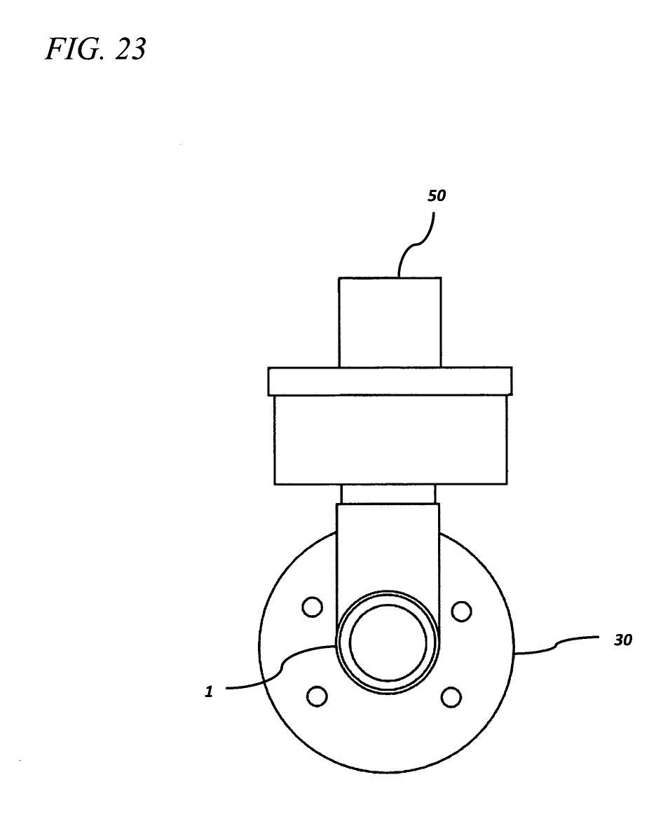

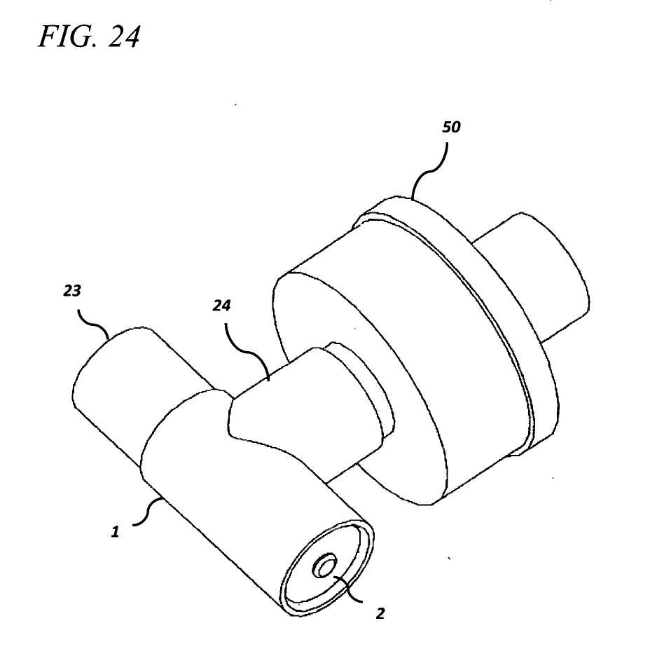

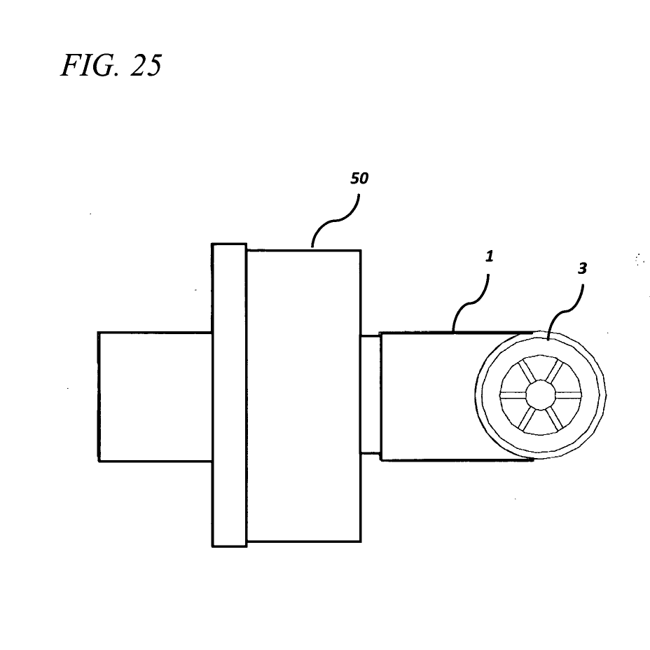

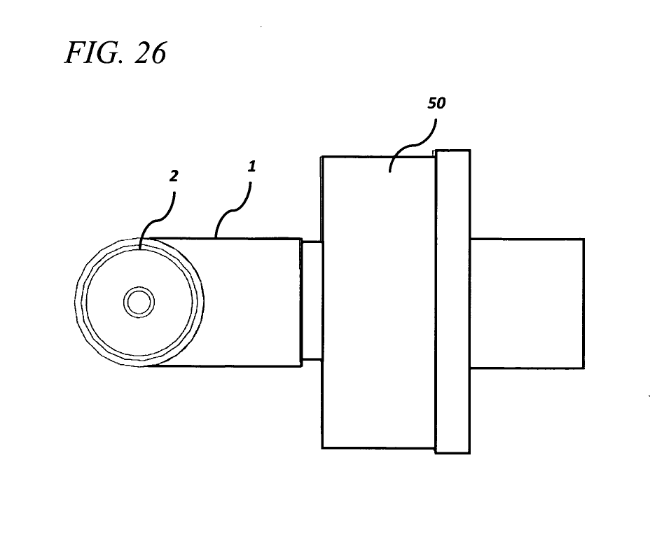

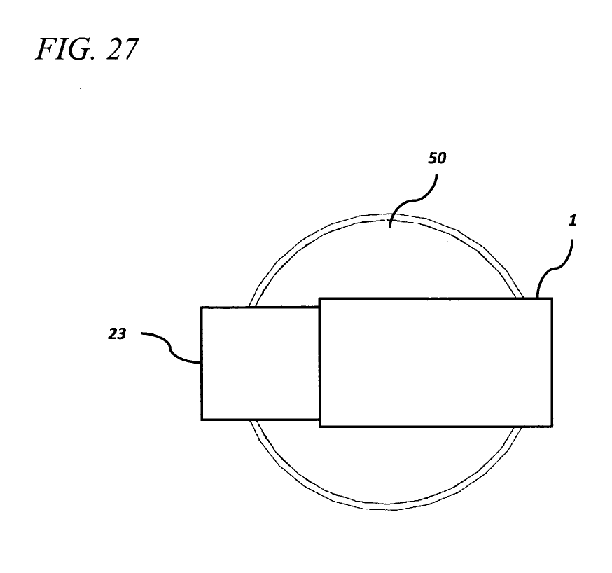

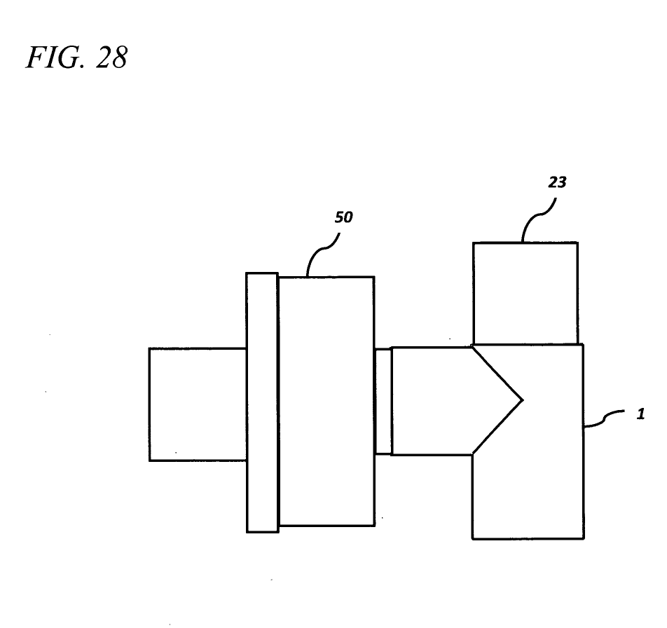

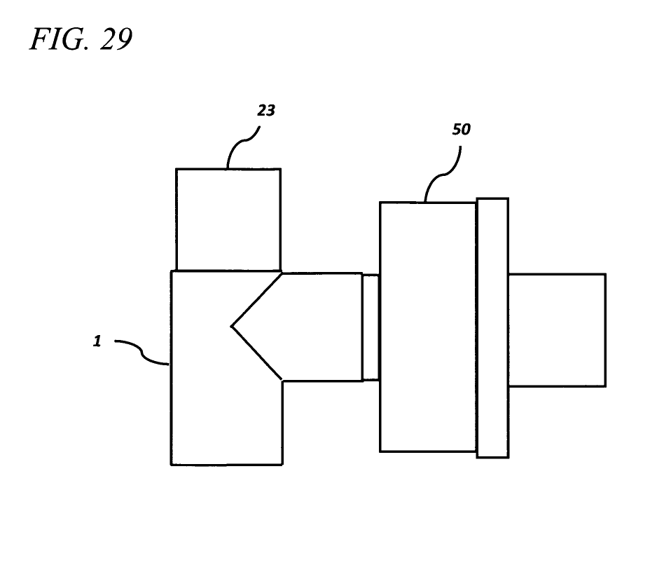

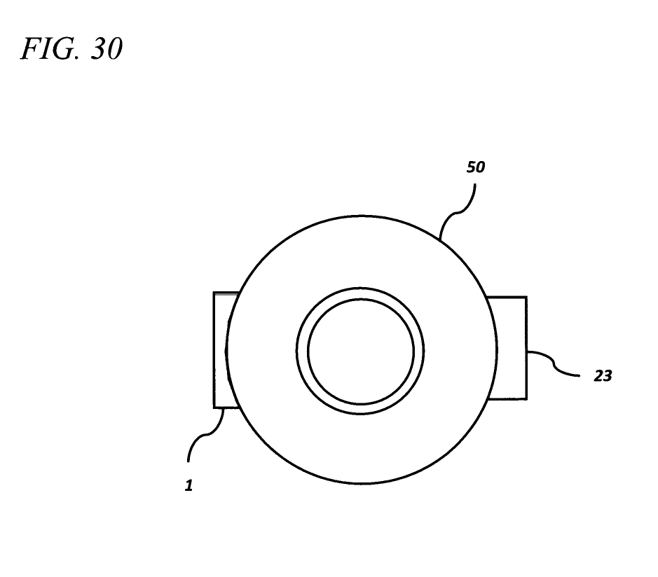

FIGS. 1 through 30 illustrate the present invention. FIG. 1 through 10 depicts a first embodiment of the present invention wherein the primary goal of achieving a valve assembly with enhanced intrathoracic pressure regulation is achieved. A valve assembly comprises a patient connector 1 in fluid communication with a dual area exhalation valve assembly 30 comprising a dual area valve base 4, wherein dual area valve base 4 has a shouldered orifice extend there through and defining a diameter and surface area of exposure. Copending from the patient connector 1 is a secondary fluidic flow-path into check valve body 2. Within check valve body 2 is located check valve flapper 3. Returning to dual area valve base 4, a dual area valve piston base 5 is affixed thereto such that dual area valve piston base 5 is influenced by respired gas pressure coming from the patient through patient connector 1, through the shouldered orifice and acting upon dual area valve piston base 5. Dual area valve piston base 5 operates by linear translation through a central aspect of dual area valve piston seal 6 and into dual area valve piston top 7, which snaps into place over the dual area valve piston seal 6 and retains said dual area valve piston seal 6 in fluid communication with dual area valve piston base 5. Acting upon dual area valve piston base is a biasing means, herein depicted as a helically wound dual area valve spring 9. Dual area valve spring 9 acts upon dual area valve piston base 5 to maintain a variable impingement upon dual area valve base 4 and thereby a tidal volume displaced by the patient acts upon the surface area of dual area valve base 5 of dual area valve piston 4. When the patient pressure exceeds the force exerted by dual area valve spring 9, the dual area valve piston base 5 will displace and allow gas to vent through the assembly. Retaining dual area valve spring 9 in place is dual area valve top 8. Dual area valve top 8 exhibits a vent rate through a plurality of atmospheric vent 25 extending through the thickness of dual area valve top 8.

Patient connector 1 may include any suitable design to allow for interfacing between the valve assembly and a patient. Preferably, patient connector 1 has a patient port 23 with an ISO respiratory fitting having a 22 millimeter outer diameter and a 15 millimeter inner diameter, so as to be readily connected to a conventional sealing-type face mask, endotracheal tube or like device.

Check valve body 2 may be comprised of any suitable composition and formed by standard techniques applicable to the medical device industry. Compositions may include metallic or non-metallic substrates, with non-metallic polymers of the thermoset and/or thermoplastic types preferred. Conventional injection molding technology can be employed in the manufacture of the check valve body 2.

Within the check valve body 2 is check valve flapper 3. Check valve flapper 3 can be any suitable material, with non-metallic polymers having a durometer between 10 and 110 being preferred. In the alternative, thin non-reactive polymeric materials such as silicon and Mylar in thicknesses of between 0.001 inch and 0.040 inch being preferred.

Dual area valve base 4, dual area valve piston base 5, dual area valve piston top 7 and valve piston top 8 may each be comprised of the same or different suitable composition and formed by standard techniques applicable to the medical device industry. Compositions may include metallic or non-metallic substrates, with non-metallic polymers of the thermoset and/or thermoplastic types preferred. Conventional injection molding technology can be employed in the manufacture of the check valve body 2. Further, dual area valve base 4 comprises an extended shoulder circumscribing a centrally located fluidic pathway having a defined internal diameter and one or more cross seal vent 22 extending through the thickness of an outer flange region of the dual area valve base 4. The cross seal vent 22 are integral to the performance of the valve assembly as the cross seal vent 22 provide sufficient resistance to flow into and against the dual area valve piston base 5. In an embodiment of the present invention, it has been determined that optimal resistance for a human patient includes four, equally spaced holes through an outer flange region of dual area valve base 4 wherein each hole is about 0.141 inch in diameter. It is within the purview of the present invention that fewer or great number of holes may be utilized such that the total surface area of the cross-seal vent 22 is within the range of about 0.04 to 0.08 square inches, with 0.058 to 0.065 square inches being preferred. Further, the number of holes, or the resulting cross sectional area of the combined number of holes, can be altered to achieve differing performance attributes in the dual area valve. For example, a smaller combined cross seal vent 22 area can be used to induce a slower rate of reset in the valve assembly and a larger combined cross seal vent 22 area can be used to induce a faster or pulsing profile as the valve assembly resets through the compression phase. It is also within the purview of the present invention that a variable vent portal can be employed in lieu of, or in conjunction with one or more static through-hole type vents. Such a variable vent portal can be mechanically or electronically adjusted through manual, semi-automated and fully automated processes.

Dual area valve piston base 5 engages upon the extended shoulder of dual area valve base 4 such that a seal is created between a face of the dual piston base and the extended shoulder. The surface area described by the internal diameter of the extend shoulder defines a closed position area. The dual area valve piston base 5 has a separate area defined by the total surface area of the piston face, which is termed the open position area. The dual area valve piston opens at higher pressures and closes at smaller pressures due to the fact that the defined closed position area (thus the area across which there is differential pressure) is less than the total surface area of the piston across which differential pressure is applied when the dual area valve piston is in the open position. The dual area valve piston base 5 is maintained against the extend shoulder of dual area valve base 4 by the force exerted by biasing means 9 (i.e. helically wound spring), though it is within the purview of the present invention that the biasing means can be mechanically or electronically adjusted through manual, semi-automated and fully automated processes. Biasing means 9 is retained in the assembly by dual area valve top 8, which itself includes a plurality of atmospheric vents. As patient pressure builds through the patient connector 1 through dual area valve base 4 and against dual area valve piston base 5, the biasing means 9 maintains dual area valve piston base 5 closed until an initial threshold pressure is exceeded. Once the pressure exceeds the force exerted by biasing means 9, the dual area valve piston base will translate from a closed to an open condition, and will remain open until sufficient pressure is dissipated as to allow biasing means 9 to return dual area valve piston base 5 to a closed position against the extended shoulder of dual area valve base 4. It is critical to note that the operation of the aforementioned valve assembly allows for an initial higher pressure to cause the piston to open and to close based on a second lower pressure being achieved. By setting the trigger pressure, the reset pressure, and the pressure decline rate from the opening pressure to the lower closing pressure based on patient parametrics, including lung capacity, the pressure with the intrathoracic region can be specifically regulated.

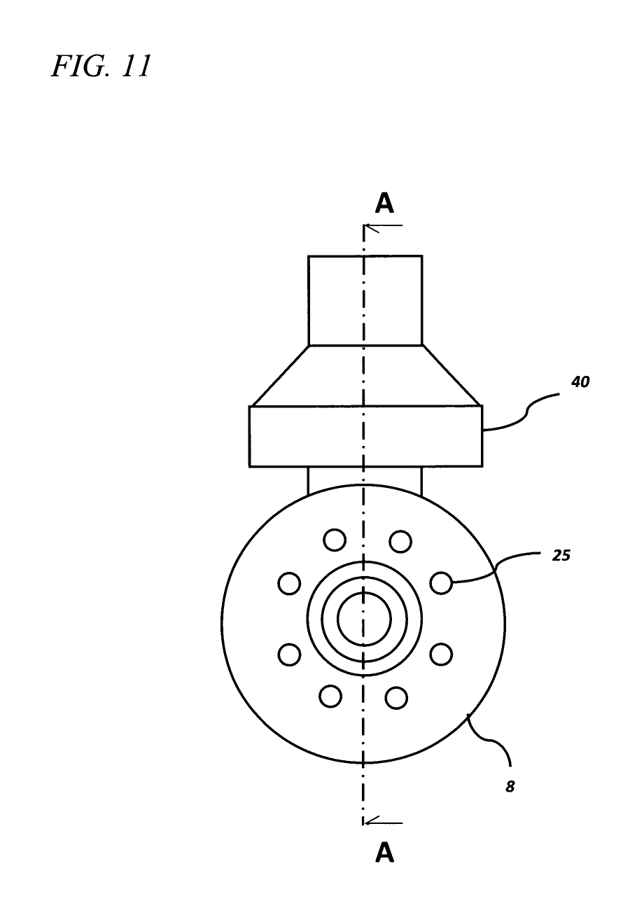

FIG. 11 through 16 depicts a second embodiment of the present invention wherein the primary goal of achieving a valve assembly with enhanced intrathoracic pressure regulation is achieved. In the second embodiment, check valve body 2 from the first embodiment is replaced with a constant vacuum valve assembly 40. Constant vacuum valve assembly 40 is comprised of a constant vacuum valve base 10 attached to a constant vacuum valve top 11. Within a spaced defined by constant vacuum valve base 10 and constant vacuum valve top 11, there is a constant vacuum valve piston 13. Constant vacuum valve piston 13 is in fluidic communication with the inhalation port 24 of the patient connector 1 and is biased into a sealed arrangement therewith by constant vacuum valve biasing means 12 (herein depicted as a helically wound spring). When a patient draws inhalation against the constant vacuum valve piston 13, negative pressure develops. When the developed negative pressure exceeds the constant vacuum valve biasing means 12, fluid is then allowed to enter. By using such a biasing control on the inhalation port 24 of the valve assembly, a negative pressure can be finitely regulated within the intrathoracic region of the patient.

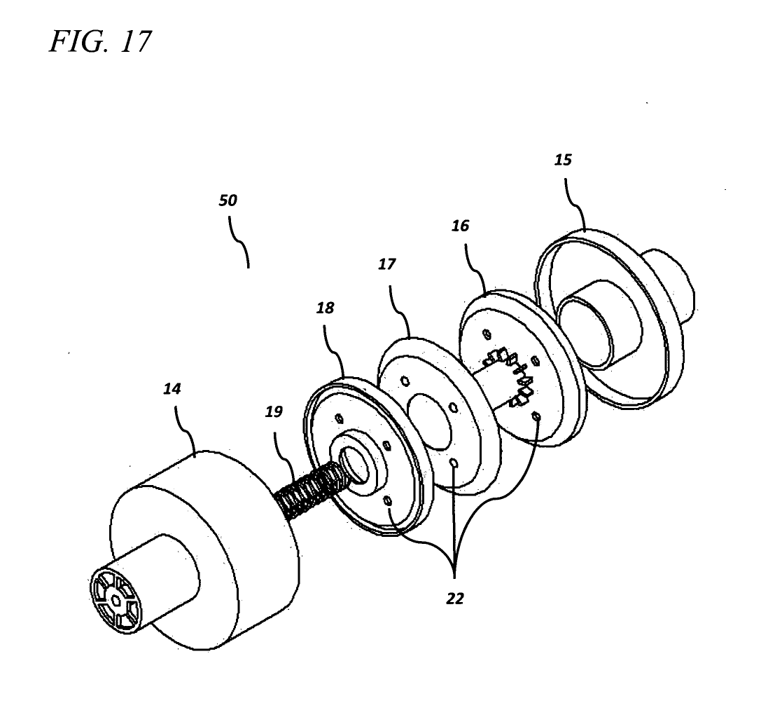

FIG. 17 through 23 depicts a third embodiment of the present invention wherein the primary goal of achieving a valve assembly with enhanced intrathoracic pressure regulation is achieved. In the third embodiment, check valve body 2 from the first embodiment is replaced with a dual area vacuum valve assembly 50. Dual area vacuum valve assembly 50 is comprised of a dual vacuum valve top 14 attached to a dual vacuum valve base 15. Within a spaced defined by dual vacuum valve top 14 and dual vacuum valve base 15, there is a dual vacuum valve piston base 16, dual vacuum valve piston seal 17, dual vacuum valve piston top 18. Dual vacuum valve piston base 16 is in fluidic communication with the inhalation port of the patient connector 1 through patient flow ports on vacuum valve top 14 and is biased into a sealed arrangement against valve base 15 therewith by dual vacuum valve biasing means 19 (herein depicted as a helically wound spring). When a patient draws inhalation against the dual vacuum valve piston base 16, an initial negative pressure develops. When the developed initial negative pressure exceeds the dual vacuum valve biasing means 19, piston base 16 is caused to move away from valve base 15, and fluid is then allowed to enter the vacuum valve. Fluid entering the vacuum valve passes through cross seal vent 22 in piston base 16 and into the internal volume of valve top 14. Valve top 14 has an extended inside diameter with a castellated top such as to arrest the initial movement of piston base 16 while also allowing fluid to flow from the internal volume of valve top 14 onto the patient flow ports that are in fluid communication with patient connector 1. Once piston base 16 opens, it will remain open until patient inhalation flow is insufficient to generate the necessary pressure across the cross seal vent 22 to overcome the force of the biasing means 19. By using such a biasing control on the inhalation port of the valve assembly, a specific set of pressures can be finitely regulated within the intrathoracic region of the patient. The opening and closing pressures of the dual area vacuum valve assembly 50 are a result of the fact that only a small portion of the dual vacuum valve piston is exposed to ambient pressure when closed, and that when the dual vacuum valve is open, significantly more area of the dual vacuum valve piston is exposed to ambient pressure, although in both cases the biasing force is about the same. The rate at which the patient vacuum pressure drops from the high opening vacuum pressure to the lower closing vacuum pressure is controlled by flow dynamics of the inhalation gas and the restrictive values of the cross seal vent 22 in the vacuum piston. Increases in the total open area of the cross vent 22 would provide for a faster decline and decreases in the total open area of the cross seal vent 22 would provide for a slower decline.

The dual area piston assembly of the present invention, whether in an exhalation or inhalation mode, exhibits significant structural and performance differences from those previously described for dual area valve pistons used in ventilatory support type devices, as exemplified by U.S. Pat. No. 6,067,984 to common inventor Piper, incorporated by reference in its entirety. Of particular note, a dual area vacuum valve 50 of the present invention differs over a dual area piston of a respiratory modulator in that in the instant invention a piston is used that has internal cross seal vent 22 and it is enclosed within a valve body which is sealed against atmospheric pressure. The cross seal vent 22 may include any suitable form of fluidic communication between the face of the piston orientated towards the patient connector 1 and the reverse face of the piston oriented towards the valve top. In practice, upon vacuum being reached by the piston assembly the rate of decline to the lower closing pressure can be modified by the degree of the restriction to flow caused by the cross-seal vent 22 in the piston. In a representative configuration the optimum size was determined to be 4 holes of 0.110 inches diameter located equidistant radially about the piston base 5. By pre-establishing the respective pressure profile of relevant components in the valve assembly, including the trigger pressure of a dual area valve piston, the reset pressure of a primary dual area valve piston assembly, and the restriction of cross-seal vent holes based on patient parametrics (including lung capacity) the pressure with the intrathoracic region can be specifically regulated.

FIGS. 17 and 24 through 30 depicts a fourth embodiment of the present invention wherein the primary goal of achieving a valve assembly with enhanced intrathoracic pressure regulation is achieved. In the fourth embodiment, check valve body 2 from the first embodiment is placed in association with the exhalation port in an opposite flow orientation such as to allow exhalation gases to escape and to impede incoming gases during inhalation. Dual area vacuum valve assembly 50 is then placed in fluidic communication with an inhalation port 24. Dual area vacuum valve assembly 50 is comprised of a dual vacuum valve top 14 attached to a dual vacuum valve base 15. Within a spaced defined by dual vacuum valve top 14 and dual vacuum valve base 15, there is a dual vacuum valve piston base 16, dual vacuum valve piston seal 17, dual vacuum valve piston top 18. Dual vacuum valve piston base 16 is in fluidic communication with the inhalation port of the patient connector 1 through cross seal vent 22 on vacuum valve top 14 and is biased into a sealed arrangement against valve base 15 therewith by dual vacuum valve biasing means 19 (herein depicted as a helically wound spring). When a patient draws inhalation against the dual vacuum valve piston base 16, an initial negative pressure develops. When the developed initial negative pressure exceeds the dual vacuum valve biasing means 19, piston base 16 is caused to move away from valve base 15, and fluid is then allowed to enter the vacuum valve. Fluid entering the vacuum valve passes through the cross seal vent 22 in piston base 16 and into the internal volume of valve top 14. Valve top 14 has an extended inside diameter with a castellated top such as to arrest the initial movement of piston base 16 while also allowing fluid to flow from the internal volume of valve top 14 onto the patient flow ports that are in fluid communication with patient connector 1. Once piston base 16 opens, it will remain open until patient inhalation flow is insufficient to generate the necessary pressure across the valve ports to overcome the force of the biasing means 19. By using such a biasing control on the inhalation port of the valve assembly, a specific set of pressures can be finitely regulated within the intrathoracic region of the patient. The opening and closing pressures of the dual area vacuum valve assembly 50 are a result of the fact that only a small portion of the dual vacuum valve piston base 16 is exposed to ambient pressure when closed, and that when the dual vacuum valve 50 is open, significantly more area of the dual vacuum valve piston base 16 is exposed to ambient pressure, although in both cases the biasing force is about the same. The rate at which the patient vacuum pressure drops from the high opening vacuum pressure to the lower closing vacuum pressure is controlled by flow dynamics of the inhalation gas and the restrictive values of the cross seal vent 22 in the vacuum piston. Increases in the total open area of the cross seal vent 22 would provide for a faster decline and decreases in the total open area of the vents would provide for a slower decline.

Returning to the dual area vacuum valve modality utilized in the present invention in the embodiments disclosed herein, it is particularly noteworthy in respect to a ventilatory type dual area valve that a dual area vacuum valve used to establish controlled intrathoracic pressure is not simply a dual area ventilatory valve connected in the reverse flow direction. In the event that a dual area ventilatory valve as presented in the aforementioned Piper '984 patent were to be connected in the reverse flow direction the result would be a valve that strictly opens during exhalation/positive patient pressure, and which the ratio of the opening and closing pressure cycling would simply be the inverse of said ratio. With regard to the exhalation dual area valve of a ventilatory device there is in addition to the biasing means of the design (in numerous commercial embodiments this biasing member is a spring), an additional biasing force in the form of ambient pressure that is unchanged by the flow configuration or connection of the valve assembly. The ambient pressure induced forces act on a ventilatory valve means from one side only, force which are opposite the direction of the biasing forces. In the case of the dual area vacuum valve assembly 50, the ambient pressure is an active force also biased in a constant direction, but varying depending on the area exposed to ambient pressure when the valve means is open or closed, thus it is possible to have a valve that opens and allows ambient air to flow in at one high vacuum pressure and closes thus preventing further ambient air from flowing in at a low vacuum pressure. Given that the dual area vacuum valve assembly 50 consists of a design biasing force 19 (normally created by a spring under compression) within the valving means closed and against ambient pressure and an closed ambient area defined as the smaller area of the valved means exposed to ambient pressure when the valve means is closed and a bigger open ambient area defined as the entire valve means area exposed to ambient pressure when the valve means is open (normally the entire valve surface), thus the valve means shall move from a closed to open position when the vacuum pressure increases to the point such that the product of the vacuum pressure (relative to ambient pressure) and the closed ambient area is equal to or greater than the biasing force. Correspondingly, the valve means shall move from an open to a closed position when the vacuum pressure decreases to the point that the product of the vacuum pressure (relative to ambient) and the open ambient area is equal to or less than the biasing force. By the operation of the dual area valve in the vacuum mode in response to pressures achieved relative to the ambient pressure, a dual area vacuum valve assembly 50 is achieved that opens at a high vacuum pressure and closes at a second, reduced vacuum pressure.

According to the present invention, methods and devices for increasing cardiopulmonary circulation induced by chest compression and decompression when performing cardiopulmonary resuscitation are provided. Such methods and devices may be used in connection with any method of CPR in which intrathoracic pressures are intentionally manipulated to improve cardiopulmonary circulation. For instance, the present invention would improve standard manual CPR, “vest” CPR, CPR with a newly described Hiack Oscillator ventilatory system which operates essentially like an iron-lung-like device, interposed abdominal compression-decompression CPR, and active compression-decompression (ACD) CPR techniques. Although the present invention may improve all such techniques, the following description will refer primarily to improvements of ACD-CPR techniques in order to simplify discussion. However, the claimed methods and devices are not exclusively limited to ACD-CPR techniques.

ACD-CPR techniques are described in detail in “Active Compression-Decompression Resuscitation: A Novel Method of Cardiopulmonary Resuscitation”, Cohen et al., American Heart Journal, 1992 124(5); “Active Compression-Decompression: A New Method of Cardiopulmonary Resuscitation”, Cohen et al., The Journal of the American Medical Association, 1992, 267(21), these incorporated by reference herein in their respective entireties.

The use of a vacuum-type cup for actively compressing and decompressing a patient's chest during ACD-CPR is described in a brochure of AMBU International A/S, Copenhagen, Denmark, entitled Directions for Use of AMBU® CardioPump™, published in September 1992. The AMBU® CardioPump™ is also disclosed in European Patent Application No. 0 509 773 A1. These references are hereby incorporated by reference.

The proper performance of ACD-CPR to increase cardiopulmonary circulation is accomplished by actively compressing a patient's chest with an applicator body. Preferably, this applicator body will be a suction-type device that will adhere to the patient's chest, such as the AMBU® CardioPump™, available from AMBU International, Copenhagen, Denmark. After the compression step, the adherence of the applicator body to the patient's chest allows the patient's chest to be lifted to actively decompress the patient's chest. The result of such active compression-decompression is to increase intrathoracic pressure during the compression step, and to increase the negative intrathoracic pressure during the decompression step thus enhancing the blood-oxygenation process and enhancing cardiopulmonary circulation. ACD-CPR techniques are described in detail in Todd J. Cohen et al., Active Compression-Decompression Resuscitation: A Novel Method of Cardiopulmonary Resuscitation, American Heart Journal, Vol. 124, No. 5, pp. 1145-1150, November 1992; Todd J. Cohen et al., Active Compression-Decompression: A New Method of Cardiopulmonary Resuscitation, The Journal of the American Medical Association, Vol. 267, No. 21, Jun. 3, 1992; and J. Schultz, P. Coffeen, et al., Circulation, in press, 1994. These references are hereby incorporated by reference.

The present invention is especially useful in connection with ACD-CPR techniques. In particular, the invention improves ACD-CPR by providing methods and devices which impede air flow into or out of the patient's lungs to enhance positive or negative intrathoracic pressure during the compression or decompression of the patient's chest, thus increasing the degree and duration of a pressure differential between the thorax (including the heart and lungs) and the peripheral venous vasculature. Enhancing intrathoracic pressure with simultaneous impedance of movement of gases into or out of the airway thus enhances venous blood flow into the heart and lungs and increases cardiopulmonary circulation.

Any of the above embodiments may further include one or more CPR aiding devices into the valve assembly, wherein visual and/or aural signals are provided to the operator of the device as to both parameters relative to effectively conducting CPR (i.e. pace or rate, measure of applied force) and patient condition (i.e. pulse, return of autonomic function/respiratory response). In addition, incorporation of a valve assembly in accordance with the present invention into a self-inflating bag-type ventilator (e.g. bag valve mask “BVM” or more commonly referred to by the name AMBU-Bag”) yields a device imminently suitable for emergency CPR situations. A representative bag valve mask is described in U.S. Pat. No. 5,163,424 which is incorporated by reference herein in its entirety.

A representative CPR bag valve mask incorporating a valve assembly in accordance with the present invention allows for the use of a finitely regulated valve assembly can be readily employed for increasing cardiopulmonary circulation induced by chest compression and decompression. The dual area valve assembly is biased against patient exhalation and any one of the described (equivalent/alternate) control means are biased against patient inhalation and thus “locked pressure windows” can be created transiently in the cycling of the positive and negative pressures formed in the intrathoracic region of a patient during the execution of the CPR method. These “locked pressure windows” are points where due to biasing of the respective control means on both the exhalation and inhalation ports of a valve assembly attached to the respiratory system of a patient, neither inhalation or exhalation can occur, and therefore a set pressure, either positive or negative, is retained with the intrathoracic region. The transient “locked pressure windows” automatically coincide with steps in the CPR method such that less force is lost in movement of air volumes and incremental pressure gains are achieved in inducing circulation of oxygenated blood in the patient. When it is a suitable time in the CPR method for introducing fresh air into the patient, a self-inflating bag component of the bag valve mask is compressed, air is forced into the patient and the CPR method compressions can immediately resume without further delay. It should be noted that either just a self-inflating bag or a self-inflating bag with additional fluidic control valving comprised therein can be used in conjunction with the valve assembly. The introduction of air being forced through the valve assembly can occur such that a tertiary valve is used such that air can be introduced without impedance by the valve assembly, wherein the tertiary valve then activates and subsequent respiratory cycling is finitely regulated by the valve assembly.

### Example

A test procedure was developed for evaluating the performance of the present invention against a no pressure management control scenario and competitive intrathoracic pressure control technologies.

An intrathoracic model was constructed by starting with two polyurethane open cell foam blocks with dimensions of 12″×12″×4″, a tensile strength of 9 psi, a density of 2.8 lbs/cubic ft, a firmness of 0.57 psi (25% deflection), and a fine cell texture type (McMasterCarr PN 8643k712). A section of foam was removed from one of the 12″×12″ faces of one of the foam blocks, hereafter referred to as the first foam block, such that a half spherical section measuring three inches in diameter by one and one half inches deep was removed from the center of the 12″×12″ face. An adjoining 2″ semi-circular conduit was then removed on the same face of the first foam block extending from the half spherical section to the mid point of one of the four edges defining the 12″×12″ face. A twelve inch length of 22 mm corrugated tubing was placed in the conduit such that one end of the 22 mm corrugated tubing was positioned in the center of the removed half spherical section and that the other end was allowed to remain free outside the perimeter of the first foam block (extending roughly 6″ there from).

A section of foam was removed from a 12″×12″ face of the remaining unmodified foam block, hereafter referred to as the second foam block, having an elliptical profile traced on said face with a primary axis of 8 inches and a secondary axis of 2 inches and a linear depth of 3 inches. Said elliptical profile was positioned upon said 12″×12″ face of second foam block such that the end of the primary axis was positioned at the edge of 12″×12″ face, 2 inches from one of the corners of 12″×12″ face with said primary axis aligned and parallel with the immediate adjacent edge of said 12″×12″ face. An additional amount of foam was removed at the point at which the primary elliptical axis made contact with an edge of the 12″×12″ face whereby a 1″×1″ conduit was created into adjoining 12″×4″ face.

A 0.5 liter hyperinflation bag (a bag with little or no elastic return for volumes up to 0.5 liters, and elastic return for volumes greater than 0.5 liters) was obtained having a stiff open end with a 22 mm ID (such as found in Mercury Medical product number 10-55800). Two 15 inch lengths of clear vinyl tubing with an OD of 7/16″ and an ID of 5/16″ were inserted into the open end of the hyperinflation bag such that one tube's end was positioned 2 inches into the hyperinflation bag and the other tube's end was inserted 6.5 inches into the hyper inflation bag. A hot-melt adhesive was used to durably and sealably affix the two vinyl tubes into the ID of the hyperinflation bag such that the only fluid communication between the ambient environment and the inside of the hyperinflation bag was by way of the two positioned vinyl tubes. A 0.030″ thick layer of liquid latex rubber was than coated over a region of one inch about the joint formed by the vinyl tubing and the immediately adjacent stiff open end of the hyperinflation bag. The coat of liquid latex rubber was allowed to dry and then recoated in exactly the same manner for a total of 6 layers.

Upon drying overnight the hyperinflation bag assembly described above was placed in the elliptical profile cavity created in the second foam block such that the hyperinflation bag was centered in the elliptical profile, the stiff open end being centered in the 1″ conduit connected to the 12″×4″ face. The elliptical cavity, containing the hyperinflation bag, was then covered with six 8″ lengths of 2″ wide high strength cloth adhesive tape such that: each edge overlapped an adjacent piece of tape; all lengths of tape were perpendicular to the primary axis of the elliptical profile; and, that each length of tape wrapped to the nearest adjacent face by at least two. Similarly the interstitial opening between the stiff open end of the hyperinflation bag and the 1″ conduit in the second foam block was covered in such that the entire interstitial opening was protected by a cloth tape multi-laminate layer.

The first foam block was then placed upon the horizontal surface of the second foam block such that the 12″×12″ face containing the half spherical space and the connecting conduit wherein in alignment such that the elliptical profile cavity was facing directly up and in such a manner that all four 4″×12″ sides of the second foam block were aligned above and coincident with the corresponding 4″×12″ sides of the first foam block. Three 60″ lengths of 2″ wide adhesive cloth taper were then wrapped circumferentially around the mating edge of the two foam blocks such that a four inch tall horizontal retentive wrap held the two foam blocks together along the entire exposed mating edges/perimeter of the two blocks.

The top and all sides of the entire assembly described immediately above were coated with approximately 0.020″-0.040″ of liquid latex rubber with particular attention and additional liquid latex rubber added to the geometric position where the 22 mm corrugated tubing and the two vinyl tubes protruded from the assembly. Liquid latex rubber was coated an additional one to two inches down the length of each of the three tubes from the point at which each protruded from the face of the foam block assembly. The assembly was allowed to sit and dry. Once dry, the assembly was turned over such that the other 12″×12″ face was facing upward and the coating process was repeated. The cycle was repeated until the entire assembly had approximately a 0.125″ thick latex shell around the entire foam assembly.

Two pieces of ¼″ thick clear acrylic pieces measuring 3″×9″ were glued together using a solvent bonding technique such that the two pieces were butt joined along their respective 9″ edges and the 3″ axis's were perpendicular. A third piece of ¼″ thick of clear acrylic measuring 1″×2″ was than similarly butt joined at the end and inside corner such that the resulting acrylic assembly made an inside corner with the 1″ edge of the third piece of acrylic adjoined to the 3″ edge of the first piece of acrylic and the 2″ edge of the third piece of acrylic adjoined to the 3″ adjacent edge of the second piece of acrylic. The acrylic assembly was than placed on the foam block assembly such that the corner immediately adjacent to the elliptical profile was fitted into the inside corner created by the acrylic assembly with such orientation that third piece of acrylic was proximal to the exit point of the vinyl tubes.

One elastic cord having a diameter of ¼″ was then wrapped around the resulting foam, latex, and acrylic assembly such that each 8″×12″ vertical face had approximately 4 to 6 wraps with each end of the elastic cord tied off to a 2 inch diameter steel ring coincident with top face of resulting assembly. Said elastic cord was repositioned and tightened until an added gas volume of 600 ml to free end of said 22 mm corrugated tubing resulted in internal pressure of 15 cm of water column pressure.

Simple 22 mm diameter flapper valves were fitted to the free ends of said 2 lengths of vinyl tubing protruding from the foam assembly such that the first flapper valve only allowed liquid to flow into said hyperinflation bag and the second flapper valve only allowed liquid to flow out of said hyperinflation bag. Additional vinyl tubing having an ID of 5/16″ and an OD of 7/16″ was attached to free ends of said flapper valves. Free end of vinyl tubing connected to the free end of first flapper valve was caused to be in fluid communication with a free standing 4 liter reservoir of liquid water at the same elevation as the foam assembly. Free end of vinyl tubing connected to second flapper valve was positioned in an open and empty jar so as to capture fluid caused to pass through simulated heart (said hyperinflation bag) during chest compressions on simulated thoracic cavity (said foam assembly) with various devices connected to simulated trachea (said 22 mm corrugated tubing).

Utilizing the previously described thoracic model, four different conditions were tested: negative control sample without pressure management means, a commercially available device in accordance with the '394 US patent to Lurie et al., a representative device in accordance with a first embodiment of the present invention and a representative device in accordance with a fourth embodiment of the present invention. The intrathoracic model was prepared for each test condition by flushing through the check valves connected to the hyperinflation bag cardiac sub-assembly for 15 minutes to remove any trapped air. Water was used to represent a blood substitute and to establish a means for determine liquid flow resulting from ten (10) consecutive chest compressions executed using each test condition. The results are presented in Table 1 below. Chest compressions were induced per U.S. National standards of one hand placed over another and full weight compression was realized at the center point of the 12″×12″ face that incorporated the elliptical profile and the simulated heart. During simulated CPR the model was placed at a height of about 40 inches above the ground. The adult male performing the CPR was of sufficient size to produce significant results having a height of 72″, a weight of 200 lbs, a shoulder size of 44 inches, and a body fat content of less than 20%.

TABLE 1
“Cardiac” Flow per Chest Compression

| | Vacuum Setting | Positive Pressure | Mean (ml flow/ | Standard Deviation |
|---|---|---|---|---|
| Test Condition | (cm-water) | Setting (cm-water) | compression) | (ml flow/compression) |
| No Pressure | n/a | n/a | 0.28 | 0.16 |
| Control Device | | | | |
| U.S. PAT No. ′394 | 10 | n/a | 0.58 | 0.08 |
| Device | | | | |
| Embodiment #3 | 15 | 20 | 0.75 | 0.05 |
| Embodiment #4 | 10 | n/a | 0.78 | 0.06 |
| Embodiment #4 | 15 | n/a | 1.01 | 0.11 |

As can be seen in Table 1, an intrathoracic pressure control means having at least one dual area valve in association with the respiratory pathway of a simulated patient receiving CPR improves flow rate by at least 1.5 times over a negative control condition and by at least 1.25 times over a continuous vacuum methodology as represented by a device in accordance with U.S. Pat. No. '394 to Lurie et al, incorporated previously by reference.

From the foregoing, it will be observed that numerous modifications and variations can be affected without departing from the true spirit and scope of the novel concept of the present invention. It is to be understood that no limitation with respect to the specific embodiments illustrated herein is intended or should be inferred. The disclosure is intended to cover, by the appended claims, all such modifications as fall within the scope of the claims.

What is claimed:

1. A device for finitely regulating intrathoracic pressure comprising;
a. a patient connector;
b. a dual area valve piston assembly;
c. a restriction;
wherein said dual area valve piston assembly is triggered to open at a first pressure;
wherein said dual area valve piston assembly is reset to close at a second pressure; and
wherein a patient connected to said device experiences a reduction in intrathoracic pressure resulting from the triggering and resetting of said device.

2. A device as in claim 1, wherein said dual area valve piston is associated with a patient exhalation port of said patient connector and said restriction is associated with a patient inhalation port of said patient connector.

3. A device as in claim 1, wherein said dual area valve piston is associated with a patient inhalation port of said patient connector and said restriction is associated with a patient exhalation port of said patient connector.

4. A device as in claim 1, wherein said first pressure is greater than said second pressure.

5. A device as in claim 1, wherein said first pressure is formed by a patient connected to said device.

6. A device as in claim 1, wherein said restriction is a valve.

7. A device as in claim 1, wherein said dual area valve piston includes a cross seal vent.

8. A device for finitely regulating intrathoracic pressure comprising;
a. a patient connector;
b. a dual area valve piston assembly;
c. a vacuum valve;
wherein said dual area valve piston assembly is triggered to open at a first pressure;
wherein said dual area valve piston assembly is reset to close at a second pressure; and
wherein said patient experiences a reduction in intrathoracic pressure resulting from the triggering and resetting of said device.

9. A device as in claim 6, wherein said first pressure is less than said second pressure.

10. A device as in claim 6, wherein said first pressure is created by a patient connected to said device.

11. A device as in claim 6, wherein said second pressure is approximately equal to an ambient atmosphere.

12. A device as in claim 6, wherein said dual area valve piston includes a cross seal vent.

13. A device as in claim 6, wherein said vacuum valve is of a constant pressure type.

14. A device as in claim 6, wherein said vacuum valve is of a dual area valve type.

15. A method for enhancing the performance of cardiopulmonary resuscitation comprising;
a. connecting a patient's respiratory pathway to a device capable of finitely regulating intrathoracic pressure;
b. performing cardiopulmonary resuscitation;
wherein said device capable of finitely regulating intrathoracic pressure comprises a dual area valve piston assembly.

16. A method as in claim 15, wherein said device capable of finitely regulating intrathoracic pressure comprises a check valve.

17. A method as in claim 15, wherein said device capable of finitely regulating intrathoracic pressure comprises a constant vacuum valve.

18. A method as in claim 15, wherein said device capable of finitely regulating intrathoracic pressure comprises a dual area vacuum valve.

19. A method as in claim 15, wherein said device is used to treat patients having compromised pulmonary performance.

20. A method as in claim 15, wherein said device is used to treat patients so as to obtain enhanced pulmonary performance.
